(19) United States
(12) Patent Application Publication
(10) Pub. No.: US 2021/0355527 A1
(43) Pub. Date: Nov. 18, 2021

(54) METHODS FOR DETECTION OF MICROBIAL NUCLEIC ACIDS IN BODY FLUIDS

(71) Applicant: Cortexyme, Inc., South San Francisco, CA (US)

(72) Inventors: Debasish Raha, South San Francisco, CA (US); Leslie J. Holsinger, South San Francisco, CA (US); Stephen S. Dominy, South San Francisco, CA (US); Casey C. Lynch, South San Francisco, CA (US)

(73) Assignee: Cortexyme, Inc., South San Francisco, CA (US)

(21) Appl. No.: 17/280,729

(22) PCT Filed: Sep. 27, 2019
(86) PCT No.: PCT/US2019/053586

§ 371 Date: Mar. 26, 2021

Related U.S. Application Data
(60) Provisional application No. 62/737,749, filed on Sep. 27, 2018.

(51) Int. Cl. C12Q 1/689; C12Q 1/686

(52) U.S. Cl. CPC C12Q 1/689; C12Q 1/686

(57) ABSTRACT

Methods for detecting microbial nucleic acids in a body fluid of a subject are provided. The methods include highly sensitive and specific procedures for detecting DNA derived from the bacteria such as Porphyromonas gingivalis, e.g., using PCR amplification and qPCR detection, in clinical and/or laboratory samples containing CSF or other biofluids.

### CROSS-REFERENCE TO RELATED APPLICATIONS

The present application claims priority to U.S. Provisional Pat. Appl. No. 62/737,749, filed on Sep. 27, 2018, which is incorporated herein by reference in its entirety.

### BACKGROUND OF THE INVENTION

Porphyromonas gingivalis is an asaccharolytic Gram-negative anaerobic bacterium that produces major virulence factors known as gingipains, which are cysteine proteases consisting of lysine-gingipain (Kgp), arginine-gingipain A (RgpA) and arginine-gingipain B (RgpB). Recently, it has been discovered that P. gingivalis can contribute to Alzheimer's disease (AD) pathogenesis through the secretion of gingipains that promote neuronal damage. Gingipain immunoreactivity in AD brains has been found to be significantly greater than in brains of non-AD control individuals. While P. gingivalis has been detected in brain tissue samples, methods for detection of P. gingivalis and diagnosis of infection in the brain and spinal cord without the need for invasive tissue biopsy have not been previously available.

### BRIEF SUMMARY OF THE INVENTION

Provided herein are methods for detecting microbial nucleic acids, such as P. gingivalis DNA, in a body fluid, such as cerebrospinal fluid or plasma, of a subject. The methods include performing an amplification reaction under conditions sufficient to selectively amplify a microbial polynucleotide (e.g., a P. gingivalis polynucleotide), and detecting the amplified polynucleotide, thereby determining that the microbial nucleic acid is present in the body fluid of the subject.

In some embodiments, the amplification includes one or more polymerase chain reactions (PCR), including one or more quantitative polymerase chain reactions (qPCR). Employing closely spaced primers in the PCR amplification steps is particularly advantageous for the detection of fragmented DNA in cerebrospinal fluid. In some embodiments, for example, the length of the amplified microbial polynucleotide is less than 400 bases, or less than 200 bases. In some embodiments, the nucleic acid to be amplified and detected is a conserved microbial gene segment, e.g., a conserved P. gingivalis gene segment such as an hmuY gene segment.

In some embodiments, the methods further include diagnosis of a microbial infection in the subject. In some embodiments, the methods further include treating the microbial infection in the subject.

### BRIEF DESCRIPTION OF THE DRAWINGS

FIG. 1 shows the performance of P. gingivalis DNA amplification by PCR, using purified P. gingivalis bacterial DNA in the presence or absence of added cerebrospinal fluid (CSF).

FIG. 2 shows that inhibitory factors present in certain CSF samples prevent detection of P. gingivalis DNA in human CSF by qPCR. Treatment of the CSF with proteinase K or heat denaturation allowed for detection of P. gingivalis DNA (16S rRNA gene fragments) from small volumes of human CSF.

FIG. 3A shows the location of primers used for amplification of P. gingivalis hmuY polynucleotide products.

FIG. 3B shows that P. gingivalis DNA is not present as large genomic fragments in CSF. Amplification of larger DNA fragments of hmuY gene of P. gingivalis was unsuccessful, but amplification of a smaller size fragment was successful.

FIG. 4A shows the qPCR detection and quantitation of P. gingivalis DNA in CSF from subjects with probable Alzheimer's disease.

FIG. 4B shows the detection and quantitation of P. gingivalis DNA by qPCR from matching saliva samples.

FIG. 4C shows an agarose gel containing PCR products produced during the measurements shown in FIG. 4A. The lower panel of this figure shows a lack of PCR products corresponding to a conserved H. pylori gene fragment.

FIG. 4D shows the age and Mini Mental Status Exam (MMSE) score on subjects and sequence identity of PCR products to P. gingivalis hmuY DNA sequence. NS=not sequenced.

FIG. 5A shows the qPCR detection above quantitation limits of P. gingivalis DNA in CSF from 50 subjects with probable Alzheimer's disease.

FIG. 5B shows sequencing data indicating the presence of P. gingivalis DNA in all subjects referenced in FIG. 5A.

### DETAILED DESCRIPTION OF THE INVENTION

The present invention provides assays for quantitative measurement of microbial nucleic acids including P. gingivalis genes such as hmuY and fragments thereof. The quantitative PCR (qPCR) methods provided herein are particularly useful for the detection of genes that are conserved between different P. gingivalis strains and other microorganisms such as P. gulae. Nested PCR assays, as described below, are highly selective and sensitive and can be used to detect P. gingivalis DNA present at low copy numbers (e.g., less than 10 copies/μL) in bodily fluids such as CSF. The use of closely spaced primers in the PCR amplification steps disclosed herein are particularly useful for identifying fragmented DNA that would otherwise escape detection. At present, the methods disclosed herein are the only known assays that allow for the detection of chronic microbial infections in the brain. The methods can be used to monitor changes in Porphyromonas species bacterial burden in the brain (e.g., during progression of conditions such as Alzheimer's disease) and other organs, and they can also be used to assess the effects of drugs that affect the survival of bacteria in the brain. The methods described herein provide simple, rapid, cost-effective, highly selective and sensitive detection of P. gingivalis and other pathogens in the brain and spinal cord.

### I. DETECTION OF P. GINGIVALIS DNA AND OTHER MICROBIAL NUCLEIC ACIDS IN BODILY FLUIDS

Disclosed herein are methods for detecting microbial nucleic acid in a body fluid of a subject. The methods include performing an amplification reaction under conditions sufficient to amplify a microbial polynucleotide, and detecting the amplified microbial polynucleotide, thereby determining that the microbial nucleic acid is present in the body fluid of the subject. The methods are particularly useful for the detection of nucleic acids present in low quantities in body fluids. For example, the methods can be used to analyze cerebrospinal fluid (CSF) and detect nucleic acids from microbes associated with oral periodontal disease such as Porphyromonas species (e.g., P. gingivalis), Treponema species (e.g., T. denticola), Tannerella forsythia, Streptococcus species (e.g., S. gordonii), Eubacterium species (e.g., E. nodatum), Fusobacterium species (e.g., F. nucleatum), Prevotella species (e.g., P. intermedia), Campylobacter species (e.g., C. rectus), Peptostreptococcus (micromonas) species (e.g., P. micros), Eikenella scorrodens, and Capnocytophaga species (e.g., C. gingivalis, C. ochracea, and C. sputigena). Identification of microbial DNA in CSF using the presently disclosed methods may indicate a microbial infection in the brain or spinal column of the subject. As described herein, P. gingivalis infection is also associated with brain conditions, including Alzheimer's disease, and other diseases. Other infectious microbes, including but not limited to, Borrelia burgdorferi and Toxoplasma gondii can also be detected using the presently disclosed methods.

A number of nucleic acid amplification reactions can be used in conjunction with the methods described herein, e.g., PCR and variations thereof (e.g., TaqMan, real time PCR, quantitative PCR), reverse transcription, strand displacement reaction (SDR), ligase chain reaction (LCR), transcription mediated amplification (TMA), or Qbeta replication. A thermally stable polymerase, e.g., Taq, can be used to avoid repeated addition of polymerase throughout amplification procedures that involve cyclic or extreme temperatures (e.g., PCR and its variants).

As used herein, the term “polymerase” refers to an enzyme that catalyzes the polymerization of nucleotides. Generally, the enzyme will initiate synthesis at the 3′-end of the primer annealed to a nucleic acid template sequence. “DNA polymerase” catalyzes the polymerization of deoxyribonucleotides in a sequence specific manner complementing the nucleic acid that the primer is annealed to resulting in a double-stranded DNA molecule. Known DNA polymerases include, but are not limited to, Thermus aquaticus (Taq) DNA polymerase.

As used herein, the term “thermostable” as applied to a polymerase means that the polymerase is able to function at high temperatures, for example 45-100° C. (e.g., 55-100° C., 65-100° C., 75-100° C., 85-100° C., or 95-100° C.), as compared, for example, to non-thermostable polymerases that can exhibit loss of activity at temperatures above around 40° C. (e.g., greater than 37° C.).

Polymerase chain reaction (PCR) is a very commonly used nucleic acid amplification technique. PCR is capable of producing large amounts of specific DNA fragments with length and sequence defined by a nucleic acid template and 5′ and 3′ primers. The essential steps include thermal denaturation of a double-stranded target nucleic acid, annealing of the primers to their complementary sequences, and extension of the annealed primers by enzymatic synthesis with DNA polymerase. Taq or another thermostable polymerase (e.g., Pfu, Paq5000, Phusion DNA polymerase) can be used. The target portion of the nucleic acid to be amplified (template) is defined by where the primers bind to the target nucleic acid. See e.g., Dieffenbach & Dveksler PCR Primer: A Laboratory Manual (Cold Spring Harbor Laboratory Press 2003).

As used herein, the terms “primers” and “oligonucleotide primer” refer to an oligonucleotide capable of acting as a point of initiation of polynucleotide synthesis (e.g., DNA synthesis) under conditions sufficient for amplification (i.e., synthesis of a primer extension product complementary to a nucleic acid strand). Typically, such conditions include the presence of one or more nucleoside triphosphates (e.g., four dNTPs) and an agent for extension (e.g., a DNA polymerase or reverse transcriptase) in an appropriate buffer and at a suitable temperature. A primer need not reflect the exact sequence of the template nucleic acid, but must be sufficiently complementary to hybridize with the template.

Quantitative PCR (qPCR) is used to amplify and simultaneously quantify one or more nucleic acid targets. The quantity can be either an absolute number of copies or a relative amount when normalized to a known DNA input (e.g., an internal or external control) or additional normalizing genes (e.g., a bacterial housekeeping gene). A number of common methods for qPCR detection involve the use of (1) non-specific fluorescent dyes that intercalate with double-stranded DNA, and/or (2) sequence-specific probe(s) labeled with a fluorescent reporter which permits detection only after hybridization of the probe (e.g., molecular beacon).

Reverse transcription can be used to amplify an RNA template. In this case, reverse transcriptase and dNTPs are included with a primed RNA molecule to produce cDNA. In embodiments where the first amplification reaction is reverse transcription, only one primer may be employed, e.g., a poly-T oligonucleotide, or a primer that hybridizes to a known sequence on the template transcript or other template RNA molecule. The single stranded cDNA produced by reverse transcription is then used for subsequent PCR. This method can be referred to as RT-PCR (reverse transcriptase PCR), not to be confused with real time PCR, which can be referred to with the same acronym. Those of skill in the art will appreciate that the level of RNA detection may not directly reflect microbial load since each microbial gene can be transcribed as multiple RNA copies in a cell.

Real time PCR and the 5′-nuclease activity of Taq DNA polymerase can be used for detecting genetic variants. The assay requires forward and reverse PCR primers that will amplify a region that includes a variant site. Variant discrimination can be achieved using FRET, and one or two allele-specific probes that hybridize to the variant site. The probes have a fluorophore linked to their 5′ end and a quencher molecule linked to their 3′ end. While the probe is intact, the quencher will remain in close proximity to the fluorophore, eliminating the fluorophore's signal. During the PCR amplification step, if the variant-specific probe is perfectly complementary to the variant allele, it will bind to the target DNA strand and then get degraded by 5′-nuclease activity of the Taq polymerase as it extends the DNA from the PCR primers. The degradation of the probe results in the separation of the fluorophore from the quencher molecule, generating a detectable signal. If the variant-specific probe is not perfectly complementary, it will have a lower melting temperature and not bind as efficiently. This prevents the nuclease from acting on the probe.

In digital PCR (dPCR, or droplet digital PCR, ddPCR), a sample is diluted and partitioned into multiple (hundreds or even millions) separate reaction chambers so that each contains one or no copies of the sequence of interest. By counting the number of positive partitions (in which the sequence is detected) versus negative partitions (in which it is not), one can determine exactly how many copies of a DNA molecule were in the original sample (see, e.g., Sykes et al. (1992) Biotechniques 13:444; Baker (2012) Nature Methods 9:541). dPCR techniques are typically conducted using nanofabricated or microfluidic devices, e.g., from Bio-Rad®, Fluidigm®, RainDance®, and Life Technologies®.

### Polymerase Chain Reaction

In some embodiments, the amplification reaction includes a polymerase chain reaction (PCR) containing one or more series of melting steps, annealing steps, and extension steps. In some embodiments, the amplification reaction includes a PCR and the method includes:

(i) combining a body fluid sample with a forward primer, a reverse primer, and a polymerase to form a PCR mixture;

(ii) conducting the PCR with the PCR mixture, wherein the PCR is conducted under conditions sufficient to amplify the microbial polynucleotide.

As described in more detail below, it has been discovered that microbial nucleic acids such as P. gingivalis DNA can be fragmented when present in cerebrospinal fluid. Accordingly, the length of polynucleotides to be amplified in the presently disclosed methods will frequently be significantly shorter than the full sequence of the relevant gene target. In some embodiments, the length of amplified microbial polynucleotide will be no longer than about 500 bases (e.g., no greater than 400 bases or no greater than 300 bases). PCR primers can be selected such that the amplified microbial polynucleotide ranges, for example, from about 75 bases to about 400 bases. PCR primers can be selected such that the length of the amplified microbial polynucleotide ranges from about 90 bases to about 165 bases, or from about 95 bases to about 160 bases, or from about 100 bases to about 155 bases, or from about 105 bases to about 150 bases, or from about 110 bases to about 145 bases, or from about 115 bases to about 140 bases, or from about 120 bases to about 135 bases, or from about 125 bases to about 130 bases. The length of the amplified microbial polynucleotide can range from about 75 bases to about 100 bases, or from about 100 bases to about 125 bases, or from about 125 bases to about 150 bases, or from about 150 bases to about 175 bases, or from about 175 bases to about 200 bases, or from about 200 bases to about 225 bases, or from about 225 bases to about 250 bases, or from about 250 bases to about 275 bases, or from about 275 bases to about 300 bases, or from about 300 bases to about 325 bases, or from about 325 bases to about 350 bases, or from about 350 bases to about 375 bases, or from about 375 bases to about 400 bases. In some embodiments, the length of a P. gingivalis polynucleotide amplified in the method ranges from about 100 bases to about 250 bases. In some embodiments, the length of a P. gingivalis polynucleotide amplified in the method ranges from about 75 bases to about 220 bases.

In some embodiments, the amplified microbial polynucleotide is a conserved microbial gene segment (e.g., a conserved P. gingivalis gene segment). As used herein, the term “conserved” refers to a sequence of nucleotides in DNA or RNA that is similar across a range of species of microbes, or across different strains in the same species of microbe (e.g., across two or more of P. gingivalis strains W83, W50, ATCC 49417, and A7A1). Examples of conserved P. gingivalis genes include, but are not limited to, hmuY, 16S rRNA, and fimA. The hmuY gene is highly conserved among P. gingivalis strains as well as in P. gulae strains. P. gulae is most abundant in dogs but has also been detected in 10-15% of dog owners (see, Yamasaki et al. Arch Oral Biol. 2012; 57(9):1183-8). In some embodiments, the conserved P. gingivalis gene segment is an hmuY gene segment. In some embodiments, the conserved P. gingivalis gene segment is a 16S rRNA segment and the length of the amplified polynucleotide is less than 400 bases.

Useful target sequences and primer sequences include, but are not limited to, sequences that are least 50% identical to any of the sequences set forth herein. The terms “identical” or percent “identity,” in the context of two or more polynucleotide sequences, refer to two or more sequences or subsequences that are the same or have a specified percentage of bases that are the same (e.g., at least 50%, 55%, 60%, 65%, 70%, 80%, 85%, 90%, 91%, 92%, 93%, 94%, 95%, 96%, 97%, 98%, 99%, or higher) over a specified region, when compared and aligned for maximum correspondence over a comparison window or designated region. In some embodiments, for example, two or more sequences may be at least 50% identical over a comparison window ranging from about 10 bases to about 2500 bases (e.g., from about 50 bases to about 1000) bases in length. In some embodiments, two or more sequences are at least 50% identical (e.g., at least 60%, 70%, 80%, or 90% identical) over a comparison window of 10-500 bases in length, or 100-500 bases in length, or 10-250 bases in length, or 100-250 in length. Alignment for purposes of determining percent sequence identity can be performed in various methods, including those using publicly available computer software such as BLAST, BLAST-2, ALIGN or Megalign (DNASTAR) software. Sequence identity can be determined, for example, using the BLAST 2.0 algorithms, which are described in Altschul et al., Nuc. Acids Res. 25:3389-3402 (1977) and Altschul et al., J. Mol. Biol. 215:403-410 (1990). In some embodiments, BLAST 2.0 can be used with the default parameters to determine percent sequence identity in conjunction with the methods described herein.

Useful target sequences further include any suitable portion of the P. gingivalis genome reported by Nelson et al. (J. Bacteriol. 2003. 185(18): 5591-5601) and can be utilized in the disclosed methods. For example, the target gene sequence can include the genes encoding Kgp, RgpA, or RgpB, or segments thereof. Additional P. gingivalis gene targets include, but are not limited to, hagA, mfa1, gyrB, dnaA, traP, DAHP, infB, tsf, prfA, rpoA, ruvA, mreB, parA, fisA, lpxA, rmlA, manA, fabD, murA, secD, surA, nrfA, etfA, sufB, nadD, groES/EL, ribE, and segments thereof.

Primers used in the disclosed methods can be selected to amplify a gene segment of interest for detecting microbial nucleic acids in the body fluid sample (e.g., a conserved P. gingivalis gene segment). In some embodiments, forward and reverse primers can be selected to amplify bases 150-400 of hmuY or a portion thereof (e.g., bases 155-395, or bases 160-390, or bases 165-385, or bases 170-380, or bases 180-375, or bases 185-370, or bases 190-365, or bases 195-360, or bases 200-355, or bases 200-350, or bases 200-345, or bases 200-340, or bases 200-335, or bases 204-330 of hmuY as set forth in SEQ ID NO:1). Alternatively, forward and reverse primers can be selected to amplify bases 100-200, or 200-300, or 300-400, or 400-500, or 500-600 of hmuY. Forward and reverse primers can be selected to amplify bases 100-300, or 200-400, or 300-500, or 400-600 of hmuY. As explained herein, the use of closely spaced primers in the PCR amplification steps disclosed herein are surprisingly useful for identifying fragmented DNA, such as P. gingivalis DNA found in cerebrospinal fluid, that would otherwise escape detection. As such, detection of fragmented microbial DNA in body fluids such as cerebrospinal fluid can be accomplishing using primers that are selected for amplification of short polynucleotides within target regions of the microbial nucleic acid. One of skill in the art will appreciate that the length and sequence of the specific primers to be employed may vary so long as the primers anneal to closely spaced sequences within the full-length target nucleic acid sequence. The primers can be selected to amplify coding regions and non-coding regions of the microbial genome of interest, or a combination of such regions. Typically, the primers will range in size from about 12 bases to about 30 bases in length (e.g., 12-15 bases, or 15-20 bases, or 20-25 bases, or 25-30 bases).

A number of body fluids can be analyzed using the methods provided herein. For example, the methods can be used for detecting P. gingivalis nucleic acids in whole blood, plasma, peripheral blood mononuclear cells, cerebrospinal fluid, urine, and/or synovial fluid. A body fluid sample can be obtained from a subject (e.g., human, rodent, canine, or other animal) by any means suitable for a clinical setting or laboratory setting. The methods provided herein are particularly useful for the identification of microbial nucleic acids that are not abundant in the body fluid under analysis. In some embodiments, the body fluid sample used in the methods is not a saliva sample. In some embodiments, the body fluid sample is a cerebrospinal fluid (CSF) sample obtained from the subject. A CSF sample may be obtained from a human subject, for example, by a lumbar puncture (spinal tap) procedure including injection of a needle (e.g., a 22 gauge needle or 24 gauge needle) into the lumbar interspace of the subject (e.g., the L2-L3 interspace, L3-L4 interspace, or L4-L5 interspace) and withdrawal of the CSF, typically around 150-500 μL or more if practical.

In some embodiments, a small portion of body fluid obtained from a subject (e.g., a person or animal) can be used directly in the PCR mixture for amplification and detection of microbial nucleic acids. In such instances, the “body fluid sample” is the body fluid itself. Alternatively, one or more nucleic acid purification steps can be conducted with the body fluid prior to the PCR(s) in the method. In these alternative cases, the “body fluid sample” is a mixture containing purified P. gingivalis nucleic acids (if present) along with other nucleic acids associated with the sample (e.g., genomic DNA or a portion thereof from the subject). The term “purified,” as used herein to refer to DNA or other nucleic acids obtained from body fluids such as CSF, means that at least a portion of other biological macromolecules (e.g., polysaccharides, polypeptides, and the like) have been removed. In some embodiments, for example, the amount of protein in a purified DNA mixture will be less than 50% of the protein present in the initial CSF sample from which the mixture is obtained. Purifying DNA from a CSF sample can therefore include removing at least 50%, 60%, 70%, 80%, 90%, 95%, or 99% of the protein present in the CSF sample. Protein concentrations can be determined by assay methods such as a Bradford assay, a Lowry assay, a BCA assay, or similar techniques. Similarly, other carbohydrate, amino acid, and lipid components can be removed from body fluids during purification of nucleic acids.

For example, proteins and other contaminants can be removed from CSF or other body fluids by extraction with phenol-chloroform or similar organic solvents, and the remaining DNA can be precipitated with an alcohol such as ethanol. Solid-phase adsorbent materials can also be employed for purification of nucleic acids from body fluid samples prior to PCR. Examples of such materials include, but are not limited to, silica or glass fibers/particles that may be used in conjunction with chaotropic agents such as sodium iodide, guanidinium isothiocyanate, and the like. Commercially available materials designed for such protocols include, but are not limited to, ILLUSTRA GFX products (GE Life Sciences), MONARCH nucleic acid purification products (New England BioLabs), QIAamp products (Qiagen), and MAG-BIND products (Omega Bio-Tek).

In some embodiments, the body fluid sample is a pre-amplified PCR mixture, which may be formed in a preliminary PCR using primers selected for pre-amplification. In some embodiments, primers are employed in a nested fashion, such that the sequence of the amplified microbial polynucleotide in the ultimate detection step resides within the sequence of the pre-amplified microbial polynucleotide resulting from the pre-amplification step. That is: (1) the 5′ end of the pre-amplification segment is upstream of 5′ end of the subsequent segment with respect to the full-length sequence, and (2) the 3′ end of the pre-amplification segment is downstream of 3′ end of the subsequent segment with respect to the full-length sequence. In some such embodiments, the method further includes:
(i-a) combining (1-a) a cerebrospinal fluid sample obtained from the subject or (1-b) DNA purified from a cerebrospinal fluid sample obtained from the subject with: (2) a forward pre-amplification primer, (3) a reverse pre-amplification primer, and (4) a polymerase to form a pre-amplification PCR mixture, and
(i-b) conducting a preliminary PCR with the pre-amplification PCR mixture, wherein the preliminary PCR is conducted under conditions sufficient to amplify a pre-amplified microbial polynucleotide (e.g., a pre-amplified P. gingivalis polynucleotide), thereby forming the pre-amplified PCR mixture;
wherein the sequence of the amplified microbial polynucleotide resides within the sequence of the pre-amplified microbial polynucleotide of step (i-b).

Primers for the pre-amplification PCR can be selected as described above. In the case of P. gingivalis hmuY as a non-limiting example of a gene target, forward and reverse pre-amplification primers can be selected to amplify bases 145-405 of hmuY or a portion thereof (e.g., bases 150-400, or bases 155-395, or bases 160-390, or bases 165-385, or bases 170-380, or bases 175-380, or bases 175-377 of hmuY). Alternatively, forward and reverse pre-amplification primers can be selected to amplify bases 90-210, or 190-310, or 290-410, or 390-510, or 490-610 of hmuY. Forward and reverse pre-amplification primers can be selected to amplify bases 90-310, or 190-410, or 290-510, or 390-610 of hmuY.

In some embodiments, the forward primer GAACGATTTGAACTGGGACA (SEQ ID NO:4) and the reverse primer AACGGTAGTAGCCTGATCCA (SEQ ID NO:5) are employed in the PCR, such that bases 204-330 of hmuY are amplified. In some embodiments where a pre-amplification step is employed, the forward pre-amplification primer GGTGAAGTCGTAAATGTTAC (SEQ ID NO:2) and the reverse pre-amplification primer TTGACTGTAATACGGCCGAG (SEQ ID NO:3) are employed in the PCR, such that bases 175-377 of hmuY are pre-amplified. In some embodiments where qPCR is employed for detection, probes annealing to bases 286-309 of hmuY may be employed; examples of such probes include, but are not limited to, /56-FAM/TTCTGTCTT/ZEN/GCCGGAGAATA CGGC/3IABkFQ/(SEQ ID NO:6).

In some embodiments, the forward primer CGAGGGGCAGCATGAT/ACTTA (SEQ ID NO:14) and the reverse primer TTGTAATATCATGCAATAAT (SEQ ID NO:15) are employed for the amplification of 16S rRNA gene segments. In some embodiments where a pre-amplification step is employed, the forward pre-amplification primer AGGATG AACGCTAGCGATAG (SEQ ID NO:12) and the reverse pre-amplification primer GTGAGCCGTTACCTCACCAAC (SEQ ID NO:13) are employed in the PCR for the pre-amplification of 16S rRNA gene segments. In some embodiments where qPCR is employed for detection of 16S rRNA gene segments, probes including, but not limited to, GCGTAACGCGTATGCAACTTGCCTTAC (SEQ ID NO:16) may be employed.

In some embodiments, the forward primer CAACCAAAGCCAAGAAGA (SEQ ID NO:20) and the reverse primer CGAAGCTGAAGTAGGAAC (SEQ ID NO:21) are employed for the amplification of Kgp gene segments. In some embodiments where a pre-amplification step is employed, the forward pre-amplification primer CTGCACTGT AATACAAGTCG (SEQ ID NO:18) and the reverse pre-amplification primer CTCAAGCCTTGGCTCACTTG (SEQ ID NO:19) are employed in the PCR for the pre-amplification of Kgp gene segments. In some embodiments where qPCR is employed for detection of Kgp gene segments, probes including, but not limited to, CACTAGCTGCCAATCCATCATT (SEQ ID NO:22) may be employed.

In certain cases, body fluids such as CSF can be treated to inactivate and/or remove sample components that would otherwise prevent successful nucleic acid amplification. For example, a protease such as proteinase K can be used to digest inhibitory protein components in the body fluid sample. Accordingly, some embodiments of the disclosed methods further include incubating the CSF sample with a proteinase prior to step (i-a) and/or step (i). In some embodiments, the proteinase is proteinase K. Proteinase K treatment can be conducted for periods of time ranging from a few minutes to an hour or more, typically at temperatures ranging from about 30° C. to about 60° C. (e.g., about 37° C. or about 56° C.). In addition, CSF samples can be heated to inactivate PCR-inhibiting sample components. In some embodiments, the method further includes heating the cerebrospinal fluid to at least about 55° C. prior to step (i-a) and/or step (i). For example, the CSF sample can be heated to about 55° C., 60° C., 70° C., 80° C., 90° C., 95° C., or 99° C. for a period of time ranging from 1-30 minutes (e.g., about 2 min, about 5 min, or about 10 min) before or after proteinase K incubation (if used) and prior to PCR amplification steps.

In some embodiments, the polymerase is a DNA polymerase. Examples of DNA polymerases include, but are not limited to, Pyrococcus furiosus (Pfu) DNA polymerase (Lundberg et al., (1991) Gene 108:1), E. coli DNA polymerase I (Lecomte and Doubleday (1983) Nucleic Acids Res. 11:7505), T7 DNA polymerase (Nordstrom et al. (1981) J. Biol. Chem. 256:3112), Thermus thermophilus (Tth) DNA polymerase (Myers and Gelfand (1991) Biochemistry 30:7661), Bacillus stearothermophilus DNA polymerase (Stenesh and McGowan (1977) Biochim Biophys Acta 475:32), Thermococcus litoralis (Tli) DNA polymerase (also referred to as Vent DNA polymerase, Cariello et al. (1991) Nucleic Acids Res 19:4193), Thermotoga maritima (Tma) DNA polymerase (Diaz and Sabino (1998) Braz J. Med. Res 31:1239), and Thermus aquaticus (Taq) DNA polymerase (Chien et al., (1976) J. Bacteoriol 127:1550).

As mentioned above, the PCR mixture of step (i) can further include a probe oligonucleotide. Suitable probes may contain a reporter dye covalently bonded to the oligonucleotide (typically at one terminus of the oligonucleotide), and one or more quencher moieties covalently bonded to other positions of the oligonucleotide (typically at the second terminus of the oligonucleotide or an internal position of the oligonucleotide). Typically, the probes will range in size from about 12 bases to about 40 bases in length (e.g., from about 22 bases to about 26 bases, or from about 20 bases to about 28 bases, or from about 20 bases to about 30 bases, or from about 15 bases to about 35 bases). Probes used in the disclosed methods are generally designed to anneal to a sequence within the amplified polynucleotide (e.g., within a conserved P. gingivalis gene segment). For example, a probe can be designed to anneal to between 12 and 40 bases within bases 150-400 of hmuY. The probe can be designed to anneal to a target region within bases 160-190 of hmuY, or within bases 190-220 of hmuY, or within bases 220-250 of hmuY, or within bases 250-280 of hmuY, or within bases 280-310 of hmuY, or within bases 310-340 of hmuY, or within bases 340-370 of hmuY.

In some embodiments, the probe is an oligonucleotide with a reporter dye bonded to the 5′ end of the oligonucleotide and a first quencher moiety bonded to the 3′ end of the oligonucleotide. In some embodiments, the probe further includes a second quencher moiety bonded at an internal position of the oligonucleotide. The second quencher can be located, for example, within 3, 4, 5, 6, 7, 8, 9, or 10 bases of the 5′ end of the oligonucleotide. Suitable reporter dyes include but are not limited to, xanthene dyes, such as fluorescein and rhodamine dyes, e.g., 6-carboxyfluorescein (FAM), 27-dimethoxy-4′5′-dichloro-6-carboxyfluorescein (JOE), tetrachlorofluorescein (TET), 6-carboxyrhodamine (R6G), N,N,N′,N′-tetramethyl-6-carboxyrhodamine (TAMRA), and 6-carboxy-X-rhodamine (ROX). Other reporter dyes include naphthylamine dyes, e.g., 5-(2′-aminoethyl)aminonaphthalene-1-sulfonic acid (EDANS)); coumarins, e.g., 3-phenyl-7-isocyanatocoumarin; acridines, e.g., acridine orange; cyanines, e.g., indodicarbocyanine 3 (Cy3) and indodicarbocyanine 5 (Cy5); Texas Red; BODIPY™ dyes; benzoxaazoles; stilbenes; pyrenes; and the like. Examples of suitable quencher moieties include, but are not limited to, aminobenzenes (e.g., dabcyl); DDQ quenchers available from Eurogentec; BHQ quenchers available from LGC Biosearch Technologies; and Iowa Black quenchers and ZEN quenchers available from Integrated DNA Technologies.

Alternatively, amplified polynucleotides can be detected and quantified using a double-stranded DNA binding or intercalating dye such as ethidium bromide, SYBR GREEN, or EVAGREEN dyes.

Instrumentation suitable for conducting the qPCR reactions of the present disclosure are available from a number of commercial sources (e.g., ABI Prism 7700, Applied Biosystems, Carlsbad, Calif.; LIGHTCYCLER 480, Roche Applied Science, Indianapolis, Ind.; Eco Real-Time PCR System, Illumina, Inc., San Diego, Calif.; RoboCycler 40, Stratagene, Cedar Creek, Tex.; CFX96 TOUCH Real-Time PCR Detection System, Bio-Rad Laboratories, Inc., Hercules Calif.).

When real time quantitative PCR is used to detect and measure the amplification products, various algorithms can be used to calculate the copy number of P. gingivalis DNA in the samples (see, for example, ABI Prism 7700 Software Version 1.7 and Lightcycler Software Version 3). Quantitation may involve the use of standard samples with known copy numbers of the nucleic acids and generation of standard curves from the logarithms of the standards and the cycle of threshold (Ct). In general, Ct is the PCR cycle where the fluorescence generated by the amplification product is several deviations above the baseline fluorescence.

### Sequencing Methods

In some embodiments, the method includes sequencing the amplified microbial polynucleotides and the generation of sequencing data. Untargeted and targeted sequencing techniques can be employed. Target-specific sequencing can include selective sequencing of specific genomic regions or specific genes. The genomic DNA of the subject can be depleted to allow focused sequencing of microbial nucleic acids as described, for example, in US 2016/0281166, which is incorporated herein by reference in its entirety. In some embodiments, detection of the pathogenic polynucleotide includes identifying a microbial DNA sequence from within the sequencing data. Sequencing data can be obtained by a variety of techniques and/or sequencing platforms. Sequencing techniques include, for example polymerase-based assays and ligase-based assays, with detection techniques such as asynchronous single molecule detection or synchronous multi-molecule detection. Alternatively, platforms which avoid amplification altogether (as described, for example, in WO 2007/025124) can be used for the sequencing of single, unamplified DNA molecules.

Several next generation sequencing technologies are available for fast and economical determination of a genome's entire sequence. Typically, a library of template nucleic acids is prepared from a genomic DNA sample prior to sequencing. In some embodiments, sample preparation can include a DNA fragmentation step that breaks the larger DNA strands into smaller DNA fragments that are more amenable to next generation sequencing technologies. It may not be necessary to conduct such fragmentation steps in the presently disclosed methods, given the fragmentation of microbial DNA discovered in body fluids (e.g., cerebrospinal fluid) as described herein. Adaptor oligonucleotides can be attached to the ends of the DNA fragments, which can be accomplished by DNA end repair followed by adaptor ligation, or by the use of transposomes. A transposome is a complex of a transposase and transposon sequences, which can provide simultaneous fragmentation and adaptor ligation of fragments thereby simplifying library preparation. Transposases, transposomes, and transposome complexes are described, for example, in U.S. Pat. Nos. 9,080,211 and 10,017,759.

Sequencing can be performed by sequencing-by-synthesis (SBS) technologies, which include the stepwise synthesis of a single strand of polynucleotide complementary to the template polynucleotide whose nucleotide sequence is to be determined. Incorporation of nucleotides can be detected by detecting the release of pyrophosphate (PPi), via chemiluminescence, or by use of detectable labels bonded to the nucleotides (e.g., mass tags, fluorescent labels, or chemiluminescent labels). Examples of SBS procedures and instrumentation are described, for example, in WO 91/06678; WO 04/018497; WO 07/123744; and U.S. Pat. Nos. 7,057,026; 7,329,492; 7,211,414; 7,315,019; 7,405,281, and 8,343,746.

Sequencing data can be generated using nanopore-based sequencing methods. In nanopore sequencing, a single-stranded DNA or RNA molecule can be electrophoretically driven through a nano-scale pore in such a way that the molecule traverses the pore in a strict linear fashion. Because a translocating molecule can partially obstruct or block the nanopore, it can alter the pore's electrical properties. This change in electrical properties can be dependent upon the nucleotide sequence, and can be measured. The nanopore can be a synthetic pore or biological membrane protein, such as alpha-hemolysin. Examples of nanopore-based methods and systems include those described in U.S. Pat. Nos. 7,001,792; 8,771,491; and 9,885,079. Sequencing can also be performed by SOLiD (Supported Oligo Ligation Detection) sequencing. The SOLiD platform can use an adapter-ligated fragment library similar to those of the other next-generation platforms, and can use an emulsion PCR approach with small magnetic beads to amplify the fragments for sequencing. The platform is described, for example, in WO 2006/084132.

Sequencing reads generated using techniques such as those described above can be compared to known sequence information, so as to identify the microbial source of the nucleic acids found in the body fluid sample. For example, the sequencing data generated in the presently disclosed methods can be compared to 16S rDNA sequence information in a database such as RDP (Ribsomal Database Project; see, Cole, et al. 2009. Nucleic Acids Res. 37, D141-D145); Greengenes (see, De Santis, et al. 2006. Appl Environ Microbiol. 72, 5069-5072); or the NCBI Nucleotide database in order to identify the microbial source of the detected DNA.

### II. DIAGNOSIS AND TREATMENT OF MICROBIAL INFECTION

In some embodiments, the methods provided herein further include diagnosing a microbial infection (e.g., a P. gingivalis infection) in the subject when it is determined that microbial nucleic acids are present in the body fluid of the subject. In some embodiments, the microbial infection is a chronic infection. As used herein, the term “chronic infection” refers to an infection that does not rapidly resolve itself. For example, a chronic infection may persist for months or years. In contrast, an “acute infection” will typically be characterized by rapid onset and resolution within a short timeframe, e.g., 14 days or less. Persistence may be established after an acute infection period involving activation of a subject's immune system. The measurable bacterial load during a chronic infection will frequently be considerably lower than the measureable bacterial load during an acute infection. In some embodiments, intact microbial cells (e.g., P. gingivalis cells) are not detectable in the body fluid sample; the methods provided herein can be particularly useful in such cases.

Diagnosis of a P. gingivalis infection in the brain can be made when as few as around 1, 2, 5, or 10 copies of a target nucleic acid (e.g., hmuY DNA) per microliter of CSF are detected. Accordingly, some embodiments according to the present disclosure include: (a) determining that a P. gingivalis gene target (e.g., hmuY) is present in cerebrospinal fluid of the subject at concentrations ranging from 10 to 100 copiers per microliter (e.g., around 10, 12, 15, 20, 25, 30, 40, 50, 60, 70, 75, 80, 90, 95, or 99 copies per microliter), and (b) diagnosing a P. gingivalis brain infection in the subject.

Diagnosis of P. gingivalis infection, in particular, can be useful for diagnosing and/or treating a number of diseases and conditions with which such infections are correlated, including Alzheimer's disease and other brain disorders, cardiovascular disease, diabetes, cancer, liver disease, kidney disease, preterm birth, arthritis, pneumonia, and other disorders. These correlations are described, for example, by Bostanci, et al. FEMS Microbiol Lett, 2012. 333(1): 1-9; Ghizoni, et al. J Appl Oral Sci, 2012. 20(1): 104-12; Gatz, et al. Alzheimers Dement, 2006. 2(2): 110-7; Stein, et al. J Am Dent Assoc, 2007. 138(10): 1314-22; quiz 1381-2; Noble, et al. J Neurol Neurosurg Psychiatry, 2009. 80(11): 1206-11; Sparks Stein, et al. Alzheimers Dement, 2012. 8(3): 196-203; Velsko, et al. PLoS ONE, 2014. 9(5): e97811; Demmer, et al. J Dent Res, 2015. 94(9S): 201-S-11S; Atanasova and Yilmaz. Molecular Oral Microbiology, 2014. 29(2): 55-66; and Yoneda, et al. BMC Gastroenterol, 2012. 12: 16.

Upon diagnosis of a P. gingivalis infection (e.g., a P. gingivalis brain infection) in a subject using the methods provided herein, further steps can be taken to reduce or eliminate the infection in the subject. For example, one or more bacteriocidal and/or bacteriostatic agents may be administered to the subject. Examples of bacteriocidal and bacteriostatic agents include, but are not limited to: quinolones (e.g., moxifloxacin, gemifloxacin, ciprofloxacin, oflaxacin, trovafloxacin, sitafloxacin, and the like), β-lactams (e.g., penicillins such as amoxicillin, amoxacilin-clavulanate, piperacillin-tazobactam, penicillin G, and the like; and cephalosporins such as ceftriaxone and the like), macrolides (e.g., erythromycin, azithromycin, clarithromycin, and the like), carbapenems (e.g., doripenem, imipenem, meropinem, ertapenem, and the like), thiazolides (e.g., tizoxanidine, nitazoxanidine, RM 4807, RM 4809, and the like), tetracyclines (e.g., tetracycline, minocycline, doxycycline, eravacycline, and the like), clindamycin, metronidazole, and satranidazole. Chlorhexidines (e.g., chlorhexidine digluconate) and zinc compounds (e.g., zinc acetate) can also be used in combination with the antibiotics.

Gingipain inhibitors can be also be used to reduce or eliminate P. gingivalis infection following diagnosis. Inhibition of Kgp, RgpA, and RgpB, which are considered narrow-spectrum virulence targets, can complement the activity of broad-spectrum antibiotic and can in certain instances help to minimize antibiotic resistance. Examples of gingipain inhibitors include, for examples, those described in WO 2016/057413, WO 2017/083433, WO 2018/053353, and WO 2018/209132, which are incorporated herein by reference in their entirety. Such compounds can inhibit active gingipains in the brain of a mammal, e.g., a human or an animal (e.g., a dog), and are cytoprotective or neuroprotective. By “neuroprotective,” it is meant that the compounds prevent aberrant changes to neurons or death of neurons. Treatment of P. gingivalis infection in the brain can therefore be used in the amelioration of brain disorders and neurodegenerative disease such as Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, cerebrovascular disease, Lewy Body disease, multiple system atrophy, schizophrenia, and depression. Accordingly, some embodiments of the present disclosure include identifying a patient demonstrating a symptom of a neurological disorder such as Alzheimer's disease, determining that a P. gingivalis infection is present in the brain of the subject, and treating the infection.

Active agents can be administered at any suitable dose. For example, a gingipain inhibitor can be administered at a dose ranging from about 0.1 milligrams to about 1000 milligrams per kilogram of a subject's body weight (i.e., about 0.1-1000 mg/kg). The dose of gingipain inhibitor can be, for example, about 0.1-1000 mg/kg, or about 1-500 mg/kg, or about 25-250 mg/kg, or about 50-100 mg/kg. The dose of gingipain inhibitor can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. Active agents such as bacteriocidal agents and bacteriostatic agents can be dosed in a similar fashion. The dosages can be varied depending upon the requirements of the patient, the severity of the disease or condition being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the disease or condition.

### III. KITS

Also provided herein are kits including components for use in detecting microbial nucleic acids (e.g., P. gingivalis DNA) in body fluid samples such as CSF. The kit can include (e.g., in separate containers, separated compartments, wells, tubes, packages, burst packs; or in contained, defined areas, e.g., dried on a surface) one or more components selected from: (i) a first set of primers specific for a microbial polynucleotide (e.g., a P. gingivalis polynucleotide); (ii) a second set of primers specific for use in a pre-amplification PCR as described above; and (iii) a detection reagent (e.g., a probe oligonucleotide). The kit can also include reagents for carrying out the amplification reaction(s) and downstream analyses, e.g., amplification enzyme(s) (Taq, reverse transcriptase, or other RNA or DNA polymerases), buffers, single nucleotide mixes, intercalating dyes, etc. The kit can also include consumables for carrying out the amplification reaction(s) and downstream analyses, e.g., multiwell plates, tubes, cuvettes, pipettes, etc. The kit can also include an amplification inhibitor, e.g., as a control to aid in setting a threshold for determining abnormality in an amplification reaction.

Diagnostic devices and kits useful for identifying microbial infections in body fluid samples can include oligonucleotides for detecting microbial nucleic acid sequences. The oligonucleotides can be attached to one or more solid substrates such as microchips and beads. For example, a diagnostic kit can include a microarray containing oligonucleotides than bind to portions of the P. gingivalis genome sequence as described by Nelson et al. (J. Bacteriol. 2003. 185(18): 5591-5601). Kits according to the present disclosure can include one, two, or more (e.g., at least 2, 3, 4, 5, 7, 8, 9, 10, 11, 12, 13, 14, 15, 16, 17, 18, 19, 20, 21, 22, 23, 24, 25, 26, 27, 28, 29, 30, 40, 45, 50, 55, 60, 65, 70, 75, 80, 85, 90, 95 or 100) sets of oligonucleotides. Each set can include one or more oligonucleotides (e.g., from about one to about 10,000, such as from 50, 100, 200 or 300 to about 10,000). Array slides or chips can be fabricated according to known methods, e.g., as described in Bowtell and Sambrook (DNA Microarrays: A Molecular Cloning Manual, 2003 Cold Spring Harbor Laboratory Press) and Bumgarner (“Overview of DNA Microarrays: Types, Applications, and Their Future.” Current Protocols in Molecular Biology. 2013, 101(1): 22.1.1-22.1.11).

### IV. EXAMPLES

### Example 1. Nucleic Acid Sample Preparation and PCR Methodology

CSF DNA Isolation. DNA was isolated from 50 μL CSF using a Bacteria/Yeast DNA Extraction Kit (Qiagen) with some modifications as detailed below. An 11× volume (550 μL) of lysis buffer was added to 50 μL CSF and the mixture was incubated at 80° C. for 5 min. Attempts to lyse P. gingivalis with a reduced volume (3×) of lysis buffer resulted in low recovery of DNA. The lysate was then incubated with 3 μL proteinase K (Qiagen) at 56° C. for 1 h followed by incubation with RNase for 15 min to digest protein and RNA respectively. Protein precipitation solution (200 μL) was added to each tube and incubated on ice for 5 min. Precipitated proteins were centrifuged at 13K for 5 min at room temperature using an Eppendorf centrifuge, model 5415R. Supernatants were transferred to fresh tubes, and 2 μL Pellet Paint (EMD Millipore) and 600 μL isopropanol were added to each tube. The samples were incubated at room temperature for 5 minutes and precipitated DNA was collected by centrifugation at 13K for 5 min. The supernatants were removed and 600 μL 70% ethanol was added to each tube. The tubes were centrifuged at 13K for 5 min prior to removal of the 70% ethanol. The final DNA pellets were dissolved in 25 μL hydration buffer.

Using an alternative method, DNA was isolated from 200 μL CSF using a MAG-BIND® cfDNA kit (Omega Bio-Tek). The CSF was combined with 300 μL of elution buffer, followed by 15 μL Proteinase K and 33.5 μL of DS buffer, and the resulting mixture was incubated at 60 C.° for 20 min. Sample tubes were cooled, and 500 μL JSB buffer mix was added to each tube and mixed. 10 μL of magnetic binding beads were added and mixed well for 10 minutes. The tubes were place on a magnetic stand and the liquid was removed; this process was repeated with 500-μL portions of GT7 buffer and SPW buffer containing ethanol. After drying the bead pellet for 25 minutes, DNA was eluted by adding 30 μL of elution buffer and vortexing for 5 minutes prior to removing the magnetic beads and transferring the DNA to fresh tubes for storage.

Nested PCR Method. CSF DNA (5 μL) was first amplified for 20 cycles (pre-amplification) with hmuY-specific primers hmuY F1.2 (5′-GGTGAAGTCGTAAATGTTAC-3′; SEQ ID NO:2) and hmuY R1.2 (5′-TTGACTGTAATACGGCCGAG-3′; SEQ ID NO:3). These primers amplify a 203 bp fragment of the hmuY gene. Pre-amplification PCR was run on a 96-well plate. Each reaction mixture contained 5 μL CSF DNA, 0.5 μM forward and reverse primers, and FastKapa PCR Mix (Kapa Biosystem) in a total volume of 20 μL. The PCR cycling conditions for the pre-amplification included steps (1)-(6). Step (1): 3 min incubation at 95° C.; step (2): 20 sec incubation at 95° C.; step (3): 2 min incubation at 60° C.; step (4): 20 sec incubation at 72° C.; step (5): 19 additional cycles of steps (2)-(4); and step (6): 10 min hold.

A quantitative PCR assay was then performed from 1 μL of the pre-amplified PCR product using hmuY-specific primers hmuY F1.1 (5′-GAACGATTTGAACTGGGACA-3′; SEQ ID NO:4) and R1.1 hmuY (5′-AACGGT AGT AGCCTGATCCA-3; SEQ ID NO:5), as well as hmuYProbe3 (5′-/56-FAM/TTCTGTCTT/ZEN/GCCGGAGAATACGG C/3IABkFQ/-3′; SEQ ID NO:6). The qPCR assay was performed on a 96-well plate. Each reaction consisted of 1 μL pre-amplification product, 0.5 μM primers and probe, and FastKapa PCR Mix in a total volume of 20 μL. The PCR cycling conditions for the pre-amplification included steps (q1)-(q6). Step (q1): 3 min incubation at 95° C.; step (q2): 30 sec incubation at 95° C.; step (q3): 15 second incubation at 55° C.; step (q4): 30 sec incubation at 60° C.; step (q5): fluorescence measurement; step (q6): 39 additional cycles of steps (2)-(5).

A synthetic DNA template (GBlock H1.2; set forth in SEQ ID NO:7) of 250 bp in length representing part of the hmuY gene sequence was designed and synthesized. The sequence of the synthetic DNA template encompassed primer sequences that were used for pre-amplification and quantitative PCR. A serial dilution of GBlock-H1.2 was processed along with CSF samples and was used to determine copy number of P. gingivalis in CSF.

### Example 2. Evaluation of P. gingivalis DNA Detection in CSF and Development of Sample Preparation Methods

Initial attempts to determine P. gingivalis copy number without CSF DNA isolation and pre-amplification steps met with limited success, which was most likely due to the low copy number of P. gingivalis in CSF and the presence of PCR inhibitory factors such as protein and hemin in relatively high concentrations. PCR amplification of serially diluted, purified P. gingivalis DNA in the presence or absence of CSF also demonstrated the inhibitory influence of such factors in CSF. PCR amplification was detected only at higher P. gingivalis DNA concentrations, and even at these concentrations the amount of PCR product was four-fold less.

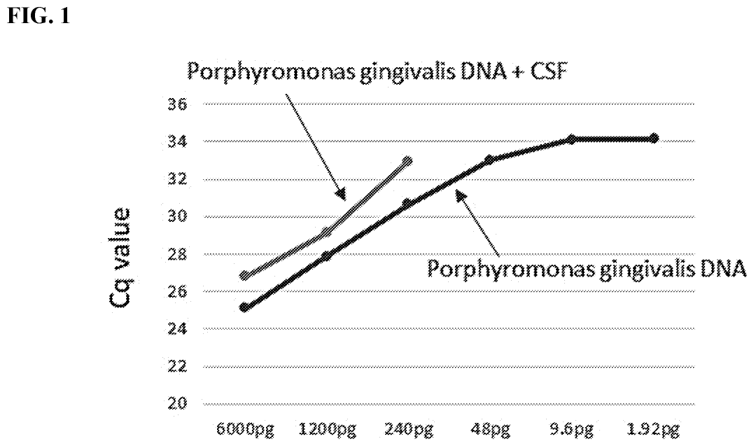

The effect of CSF inhibitory factors on PCR was quantified by mixing CSF with known quantities of purified P. gingivalis DNA before amplification. 6000 picograms of purified P. gingivalis DNA was mixed with 2 μL CSF and a 5-fold dilution series was prepared. PCR performance of P. gingivalis DNA+CSF samples was compared with same quantity of P. gingivalis DNA without any CSF added, as shown in FIG. 1. The data indicate less efficient (higher Cq values) amplification of P. gingivalis DNA in samples containing CSF at higher DNA concentrations and lack of any amplification at all, in samples with lower DNA concentrations. The inhibitory effect remained strong even after dilution of CSF several fold. 16S gene specific primers (For: 5′-ACGAGTATTGCATTGAATG-3′, SEQ ID NO:8; and Rev: 5′-ACCCTTTAAACCCAATAAATC-3′, SEQ ID NO:9) were used for PCR amplification.

The experiments demonstrated that PCR amplification is blocked by the inhibitory factors in CSF particularly when samples contain low levels of P. gingivalis DNA. Simple dilution did not overcome the observed inhibition, so additional purification and treatment methods were investigated.

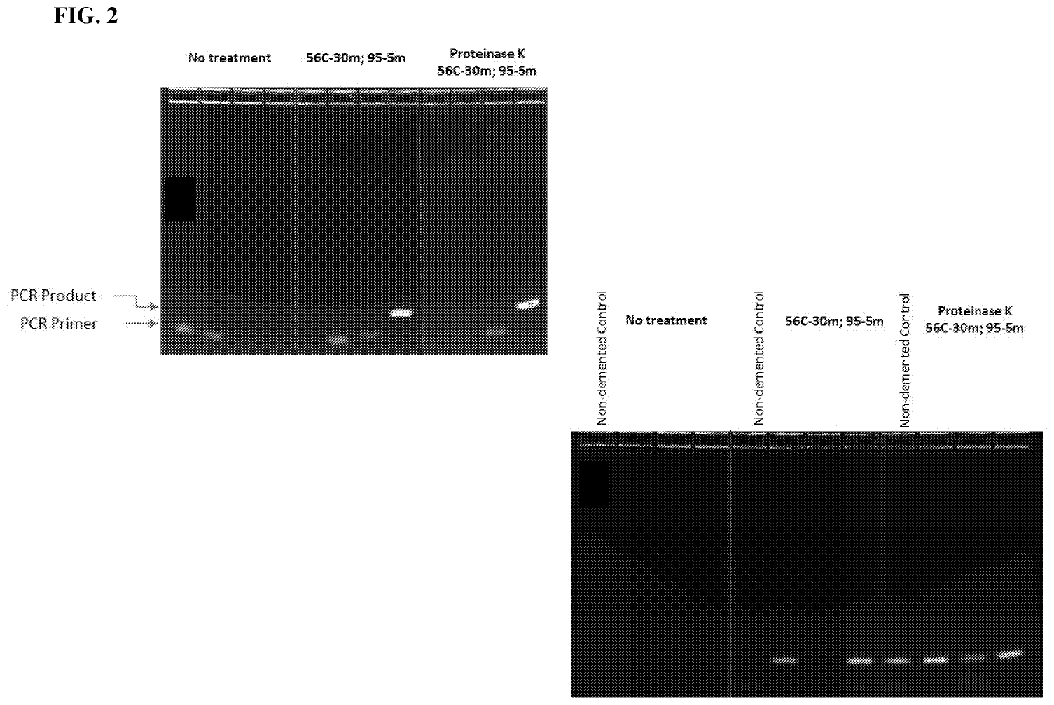

Some amplification was observed when CSF samples were treated with proteinase K (56° C. for 1 h) or denatured with heat (95° C. for 5 min) prior to PCR cycling. Agarose gels with and without PCR products generated from amplification of 0.2-2.0 μL CSF with 16S primers (For: 5′-ACGAGTATTGCATTGAATG-3′, SEQ ID NO:10; and Rev: 5′-ACCCTTTAAACCCAATAAATC-3′, SEQ ID NO:11) are shown in FIG. 2. The left panel of the first gel shows that P. gingivalis in CSF without any treatment (“No Treatment”) did not lead to the identification of a PCR product, indicating the presence of inhibitory factors. A partial success in detecting P. gingivalis DNA by PCR was obtained when CSF was treated with proteinase K followed by exposure to 95° C. for 5 min (FIG. 2, first gel, right panel). The PCR was successful only with smallest quantity (0.2 μL) of protease-treated CSF indicating incomplete removal of inhibitory substances. Heat treatment alone (FIG. 2, first gel, middle panel) was similarly helpful. To determine the reproducibility of the effect of treatment with proteinase K or heat, the treatments were tested on three more CSF samples using PCR products from amplification of 0.2 μL CSF. Amplification from CSF of all subjects was observed when samples were subjected to both proteinase K treatment and heat treatment (FIG. 2, second gel). Despite the improvements in detection, the maximum input of protease- and heat-treated CSF in the PCR was still limiting, and low copy numbers of P. gingivalis present in the samples limited reproducibility of these initial attempts.

Use of purified DNA prepared with DNA extraction kits as described above improved DNA detection and assay reproducibility. When DNA was prepared using 200-μL CSF samples and magnetic beads as described above, the higher amount of input DNA in the PCR reactions allowed for the detection of P. gingivalis DNA without a pre-amplification step. In certain instances, samples that tested negative when the Bacteria/Yeast DNA Extraction Kit was used for purification were found to contain P. gingivalis DNA when the MAG-BIND® cfDNA kit was used for purification.

### Example 3. P. gingivalis DNA in CSF is Fragmented

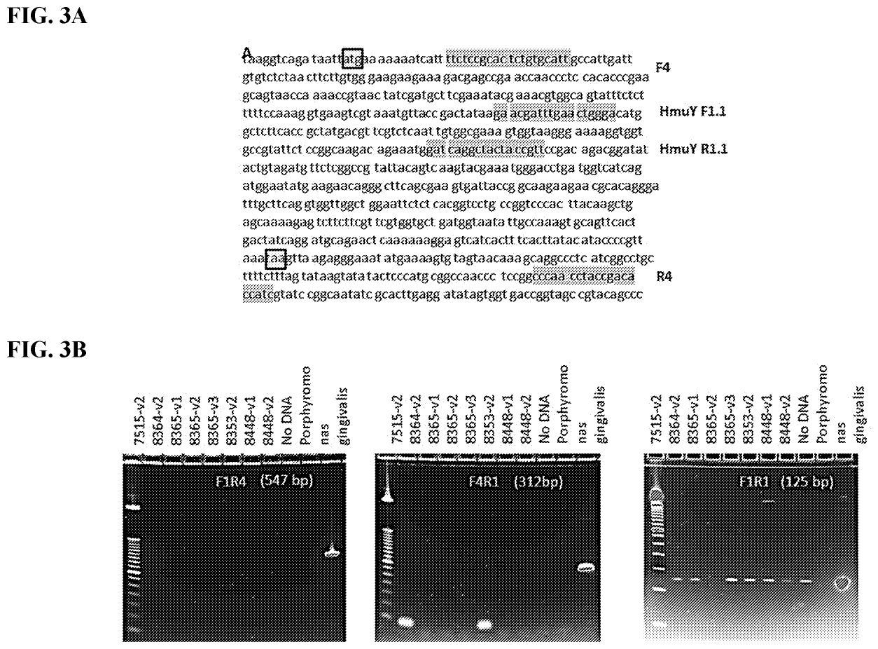

Integrity of the P. gingivalis DNA was discovered to be an important factor for efficient PCR amplification and quantification. The efficiency of amplification on amplicons of three different lengths (547 bp, 312 bp, and 125 bp) in eight CSF samples was tested. Specially-designed primers for detecting the hmuY gene, which is highly specific to Porphyromonas species, were used in this series of experiments, rather than primers for the 16S rRNA gene as used in the previous example, which is relatively conserved among prokaryotes. FIG. 3A shows the sequence of the hmuY gene, where the start and stop codons are highlighted with boxes and the sequence of four primer targets are highlighted with grey shading. FIG. 3B shows images of agarose gels with and without PCR products generated by different primer combinations. A P. gingivalis DNA control (positive) and a “no DNA” control (negative) were run along with the CSF samples for all three primer combinations.

Only the F1R1 PCR product, a 125 bp DNA fragment, was detectable from CSF (FIG. 3B, right panel). Failure to detect PCR products of 312 bp (FIG. 3B, middle panel) and 547 bp (FIG. 3B, left panel) indicated that P. gingivalis DNA was highly fragmented in the CSF samples. The P. gingivalis DNA was not present as large genomic fragments. As such, it is unlikely that that the detected DNA is derived from bacterial genomes that have remained intact. The fact that amplification of smaller gene fragments is successful while the amplification of larger gene fragment is unsuccessful is important for accurate detection, because incorrect or poorly placed primer sets will be unable to amplify the small fragments and will result in no detection.

The identity of the DNA amplified by CSF PCR assay was confirmed by sequencing. A qPCR assay was performed on five CSF samples, and the final PCR products were run on a 4% agarose gel. DNA bands were cut out from gel, and the DNA was extracted and sequenced. All PCR products obtained with this method from 5 independent CSF donor samples demonstrated at least 98% or greater identity with P. gingivalis hmuY gene sequence, verifying the specificity of this method.

### Example 4. Detection of P. gingivalis in CSF and Oral Biofluids from Subjects with Alzheimer's Disease

Detection and quantitation of P. gingivalis DNA by the nested qPCR method described herein, using 16S-, hmuY-, or kgp-specific primers, was conducted with CSF samples obtained from subjects with probable Alzheimer's disease.

16S-specific primers used for pre-amplification were 16S For1.2 (5′-AGGATG AACGCTAGCGATAG-3; SEQ ID NO:12) and 16S Rev1.2 (5′-GTGAGCCGTTACCT CACCAAC-3′; SEQ ID NO:13). The nested 16S-specific primers were 16S For1.1 (5′-CGAGGGGCAGCATGAT/ACTTA-3; SEQ ID NO:14) and 16S Rev1.1 (5′-TTGTAA TATCATGCAATAAT-3′; SEQ ID NO:15) and the 16S-specific probe oligo was 16S Probe (5′-GCGTAACGCGTATGCAACTTGCCTTAC-3; SEQ ID NO:16). A 16S GBlock having the sequence set forth as SEQ ID NO:17 was employed.

The kgp-specific primers used for pre-amplification were Kgp For1.2 (5′-CTGCA CTGTAATACAAGTCG-3′; SEQ ID NO:18) and Kgp Rev1.2 (5′-CTCAAGCCTTGGCTC ACTTG-3′; SEQ ID NO:19). The nested kgp-specific primers were Kgp For1.1 (5′-CAACC AAAGCCAAGAAGA-3′; SEQ ID NO:20) and Kgp Rev1.1 (5′-CGAAGCTGAAGTAGG AAC-3′; SEQ ID NO:21) and the kgp-specific probe oligo was Kgp Probe (5′-CACTAGCTG CCAATCCATCATT-3′; SEQ ID NO:22). A kgp GBlock having the sequence set forth as SEQ ID NO:23 was employed

DNA was isolated from CSF of eight AD subjects. Cq values obtained by qPCR indicated the presence of the P. gingivalis 16S gene (which is present as four copies in the bacteria) in all subjects and the P. gingivalis Kgp gene in five of eight subjects. Gel electrophoresis of the 16S PCR products indicated the presence of DNA fragments at or close to the expected size. The sequencing of all 16S PCR products confirmed their identity as P. gingivalis 16S fragments. The qPCR data (Cq values) and signal intensity of the major Kgp PCR product on gel matched well. The absence of detectable Kgp gene sequences in three of the AD subjects most likely reflects low levels of target nucleic acids and/or the diversity in the Kgp gene sequence among Porphyromonas species and possibly among P. gingivalis strains. Together these data indicate that while fragmented DNA from other genes of P. gingivalis can be detected, the highly conserved nature of the hmuY gene and the high level of sensitivity obtained using primers specific for the hmuY gene provide exceptional sensitivity and selectivity in the detection of P. gingivalis nucleic acids in CSF.

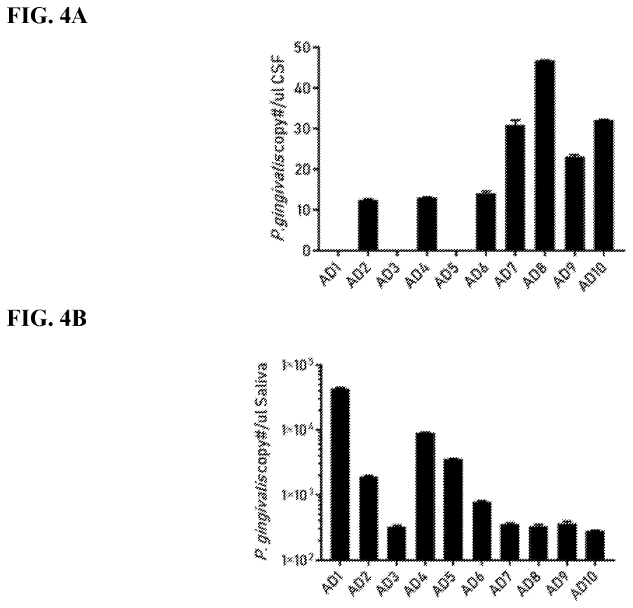

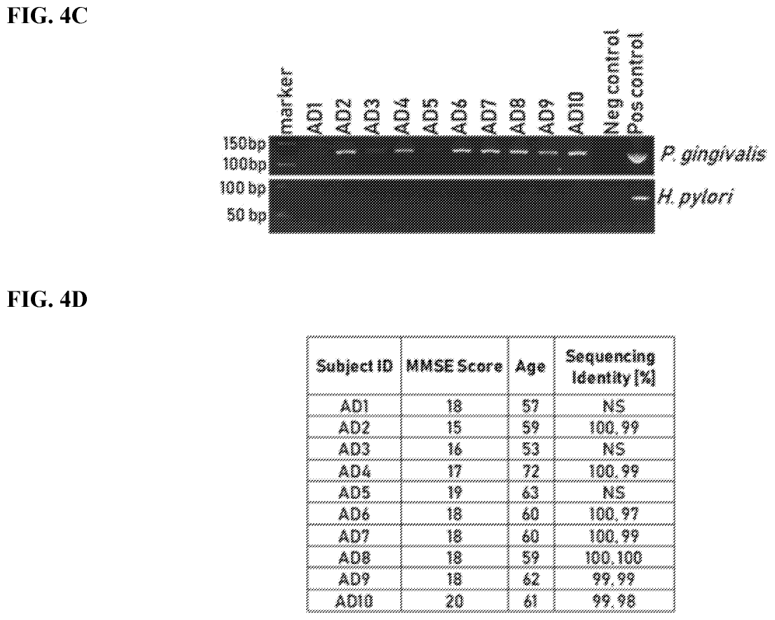

FIG. 4A shows the P. gingivalis copy number determined in the CSF samples using hmuY-specific primers, and FIG. 4B shows P. gingivalis copy number determined in saliva samples obtained from the same subjects using hmuY-specific primers. An agarose gel image of the PCR products detecting P. gingivalis from the CSF samples is shown in FIG. 4C (top panel). A negative-control and a positive control containing a synthetic DNA template were also included in the analysis. Faint or undetectable PCR products from subjects AD1, AD5, and AD5 were below the limit of quantitation for copy number and not of sufficient quantity for sequence analysis. Results from a similar assay screening for H. pylori bacteria, used as a negative control for the very sensitive amplification method, in the CSF samples is also shown in FIG. 4C (bottom panel). The table in FIG. 4D includes the age and Mini Mental Status Exam (MMSE) score on subjects and sequence identity of PCR products to the P. gingivalis hmuY DNA sequence. NS=not sequenced. These data demonstrate the reproducibility and robustness of the assay on an additional set of samples from subjects with probable Alzheimer's disease.

The same nested PCR procedure did not lead to detection of H. pylori bacteria DNA in the same samples using primers specific for a conserved gene in H. pylori, although a positive control H. pylori template spiked into CSF was able to be detected. H. pylori has been implicated in the pathology of at least one CNS disease (see, e.g., McGee, et al. Journal of Parkinson's Disease, 2018; 8 (3): 367). The absence of H. pylori in the CSF of the AD subjects indicates that the assay is specific, and that other bacterial pathogens that may also be present in the brain and have been associated with some neurological disease pathologies were not identified in the CSF of these AD subjects using this highly sensitive method.

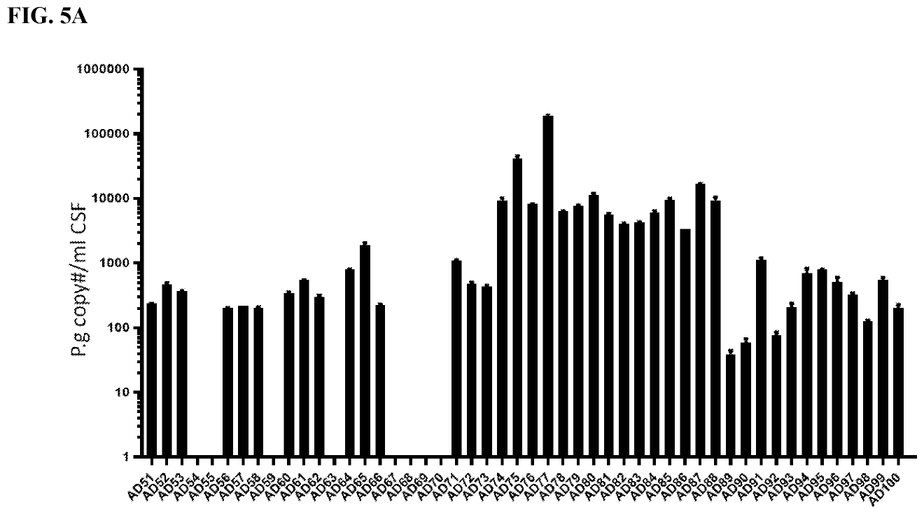

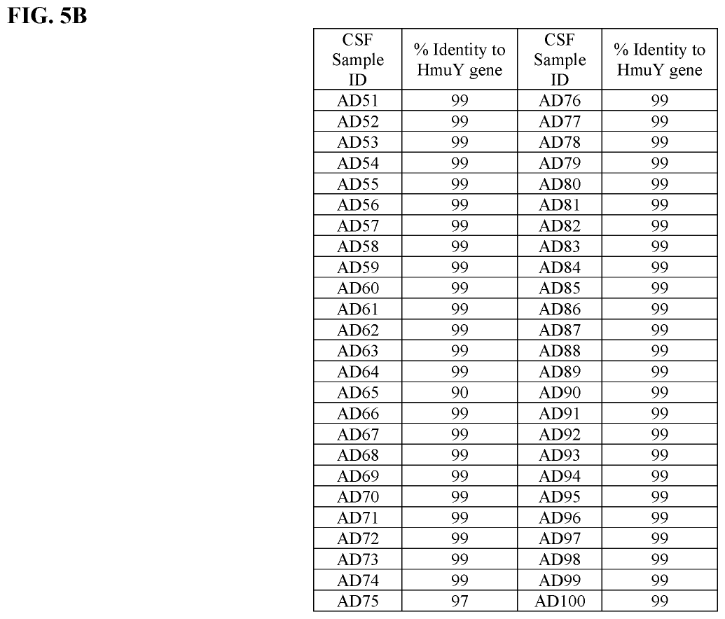

The copy number of P. gingivalis present in the CSF samples was determined by interpolation of standards. P. gingivalis was detected in 43 of 50 samples (FIG. 5A). No copy number values were obtained for 7 of 50 samples because of low abundance of P. gingivalis DNA. PCR products were separated on 4% agarose gel, prior to excision of DNA bands from the gel, extraction of DNA using a QIAquick Gel Extraction Kit (50) (Cat #28704), and sequencing of the eluted DNA. Percent identity to the P. gingivalis hmuY gene was determined using the BLAST (NCBI) protocol. Sequence data confirmed the presence of P. gingivalis DNA in all samples (FIG. 5B), including low levels in the seven samples for which no copy number value was obtained from interpolation of standards. Alignment of the sequences derived from the PCR products to P. gingivalis genome confirmed the identity of the PCR products.

### V. EXEMPLARY EMBODIMENTS

Exemplary embodiments provided in accordance with the presently disclosed subject matter include, but are not limited to, the claims and the following embodiments:
1. A method for detecting microbial nucleic acid in a body fluid of a subject, the method comprising:
performing an amplification reaction under conditions sufficient to amplify a microbial polynucleotide, and
detecting the amplified microbial polynucleotide, thereby determining that the microbial nucleic acid is present in the body fluid of the subject.
2. The method of embodiment 1, wherein the microbial nucleic acid is an oral pathogen nucleic acid.
3. The method of embodiment 1 or embodiment 2, wherein the oral pathogen is P. gingivalis.
4. The method of any one of embodiments 1-3, wherein the microbial nucleic acid comprises P. gingivalis DNA.
5. The method of any one of embodiments 1-4, wherein the length of the amplified microbial polynucleotide is less than 400 bases.
6. The method of any one of embodiments 1-5, wherein the length of the amplified microbial polynucleotide ranges from about 75 bases to about 250 bases.
7. The method of any one of embodiments 1-6, wherein the length of the amplified microbial polynucleotide ranges from about 75 bases to about 220 bases.
8. The method of any one of embodiments 1-7, wherein the amplified microbial polynucleotide is a conserved microbial gene segment.
9. The method of embodiment 8, wherein the conserved microbial gene segment is a P. gingivalis hmuY gene segment.
10. The method of embodiment 8, wherein the conserved microbial gene segment is a 16S rDNA segment less than 400 bases in length.
11. The method of any one of embodiments 1-10, wherein performing the amplification reaction comprises:
(i) combining a body fluid sample with a forward primer, a reverse primer, and a polymerase to form a polymerase chain reaction (PCR) mixture;
(ii) conducting a PCR with the PCR mixture,
wherein the PCR is conducted under conditions sufficient to amplify the microbial polynucleotide.
12. The method of embodiment 11, wherein the forward primer and the reverse primer are selected such that the length of the amplified microbial polynucleotide is less than 400 bases.
13. The method of embodiment 11, wherein the forward primer and the reverse primer are selected such that the length of the amplified microbial polynucleotide ranges from about 75 bases to about 250 bases.
14. The method of embodiment 11, wherein the forward primer and the reverse primer are selected such that the length of the amplified microbial polynucleotide ranges from about 75 bases to about 220 bases.
15. The method of any one of embodiments 11-14, wherein the body fluid sample is a cerebrospinal fluid sample obtained from the subject.
16. The method of any one of embodiments 11-14, wherein the body fluid sample comprises DNA purified from a cerebrospinal fluid sample obtained from the subject.
17. The method of any one of embodiments 11-14, wherein the body fluid sample is not a saliva sample.
18. The method of any one of embodiments 11-14, wherein the body fluid sample is a pre-amplified PCR mixture;
wherein the method further comprises:
(i-a) combining (1-a) a cerebrospinal fluid sample obtained from the subject or (1-b) DNA purified from a cerebrospinal fluid sample obtained from the subject with: (2) a forward pre-amplification primer, (3) a reverse pre-amplification primer, and (4) a polymerase to form a pre-amplification PCR mixture, and
(i-b) conducting a preliminary PCR with the pre-amplification PCR mixture, wherein the preliminary PCR is conducted under conditions sufficient to amplify a pre-amplified microbial polynucleotide, thereby forming the pre-amplified PCR mixture; and
wherein the sequence of the amplified microbial polynucleotide of step (ii) resides within the sequence of the pre-amplified microbial polynucleotide of step (i-b).
19. The method of embodiment 18, wherein:
the forward pre-amplification primer and the reverse pre-amplification primer are selected such that the length of the pre-amplified microbial polynucleotide of step (i-b) ranges from about 75 bases to about 250 bases; and
the forward primer and the reverse primer are selected such that the length of the amplified microbial polynucleotide of step (ii) ranges from about 100 bases to about 200 bases.
20. The method of any one of embodiments 15-18, further comprising incubating the cerebrospinal fluid sample with a proteinase prior to at least one of steps (i) and (i-a).
21. The method of embodiment 20, wherein the proteinase is proteinase K.
22. The method of any one of embodiments 15-21, further comprising heating the cerebrospinal fluid to at least about 55° C. prior to at least one of steps (i) and (i-a).
23. The method of any one of embodiments 11-22, wherein the polymerase is a DNA polymerase.
24. The method of any one of embodiments 11-23, wherein the PCR mixture of step (i) further comprises a probe oligonucleotide.
25. The method of any one of embodiments 1-23, wherein detecting the amplified microbial polynucleotide comprises sequencing the amplified microbial polynucleotide.
26. The method of any one of embodiments 1-25, further comprising diagnosing a microbial infection in the subject when it is determined that the microbial nucleic acid is present in the body fluid of the subject.
27. The method of embodiment 26, wherein the microbial infection is a brain infection.
28. The method of embodiment 26 or embodiment 27, wherein the microbial infection is a chronic infection.
29. The method of any one of embodiments 26-28, further comprising administering an active agent for treating the infection to the subject.
30. The method of embodiment 29, wherein the active agent is a bacteriocidal agent or a bacteriostatic agent.
31. The method of embodiment 29, wherein the active agent is a gingipain inhibitor.
32. The method of any one of embodiments 1-31, wherein intact microbial cells are not detectable in the body fluid.
33. A kit for detection of microbial nucleic acid in a body fluid sample, the kit comprising one or more components selected from: (i) a set of oligonucleotide primers for amplification of a microbial polynucleotide; and (ii) a detection reagent.
34. The kit of embodiment 33, wherein the oligonucleotide primers are selected for amplification of a microbial polynucleotide less than 400 bases in length.
35. The kit of embodiment 34, wherein the microbial polynucleotide is a P. gingivalis gene segment.
36. The kit of embodiment 34, wherein the microbial polynucleotide is a P. gingivalis hmuY gene segment.
37. The kit of any one of embodiments 33-36, further comprising (iii) a set of oligonucleotide primers for pre-amplification of a microbial polynucleotide.
38. The kit of any one of embodiments 33-37, for use in the detection of microbial nucleic acid in a cerebrospinal fluid sample.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

INFORMAL SEQUENCE LISTING

SEQ ID NO: 1 (hmuY)
ATGAAAAAAATCATTTTCTCCGCACTCTGTGCATTGCCATTGATTGTGTC
TCTAACTTCTTGTGGGAAGAAGAAAGACGAGCCGAACCAACCCTCCACAC
CCGAAGCAGTAACCAAAACCGTAACTATCGATGCTTCGAAATACGAAACG
TGGCAGTATTTCTCTTTTTCCAAAGGTGAAGTCGTAAATGTTACCGACTA
TAAGAACGATTTGAACTGGGACATGGCTCTTCACCGCTATGACGTTCGTC
TCAATTGTGGCGAAAGTGGTAAGGGAAAAGGTGGTGCCGTATTCTCCGGC
AAGACAGAAATGGATCAGGCTACTACCGTTCCGACAGACGGATATACTGT
AGATGTTCTCGGCCGTATTACAGTCAAGTACGAAATGGGACCTGATGGTC
ATCAGATGGAATATGAAGAACAGGGCTTCAGCGAAGTGATTACCGGCAAG
AAGAACGCACAGGGATTTGCTTCAGGTGGTTGGCTGGAATTCTCTCACGG
TCCTGCCGGTCCCACTTACAAGCTGAGCAAAAGAGTCTTCTTCGTTCGTG
GTGCTGATGGTAATATTGCCAAAGTGCAGTTCACTGACTATCAGGATGCA
GAACTCAAAAAAGGAGTCATCACTTTCACTTATACATACCCCGTTAAATA
A
SEQ ID NO: 2 (hmuY F1.2)
GGTGAAGTCGTAAATGTTAC
SEQ ID NO: 3 (hmuY R1.2)
TTGACTGTAATACGGCCGAG
SEQ ID NO: 4 (hmuY F1.1)
GAACGATTTGAACTGGGACA
SEQ ID NO: 5 (R1.1 hmuY)
AACGGTAGTAGCCTGATCCA
SEQ ID NO: 6 (hmuY Probe3)
/56-FAM/TTCTGTCTT/ZEN/GCCGGAGAATACGGC/3IABkFQ/
SEQ ID NO: 7 (GBlock H1.2)
AAACGTGGCAGTATTTCTCTTTTTCCAAAGGTGAAGTCGTAAATGTTACC
GACTATAAGAACGATTTGAACTGGGACATGGCTCTTCACCGCTATGACGT
TCGTCTCAATTGTGGCGAAAGTGGTAAGGGAAAAGGTGGTGCCGTATTCT
CCGGCAAGACAGAAATGGATCAGGCTACTACCGTTCCGACAGACGGATAT
ACTGTAGATGTTCTCGGCCGTATTACAGTCAAGTACGAAATGGGACCTGA
SEQ ID NO: 8 (16S For)
ACGAGTATTGCATTGAATG
SEQ ID NO: 9 (16S Rev)
ACCCTTTAAACCCAATAAATC
SEQ ID NO: 10 (16S For)
ACGAGTATTGCATTGAATG
SEQ ID NO: 11 (16S Rev)
ACCCTTTAAACCCAATAAATC
SEQ ID NO: 12 (16S For1.2)
AGGATGAACGCTAGCGATAG
SEQ ID NO: 13 (16S Rev1.2)
GTGAGCCGTTACCTCACCAAC
SEQ ID NO: 14 (16S For1.1)
CGAGGGGCAGCATGAT/ACTTA
SEQ ID NO: 15 (16S Rev1.1)
TTGTAATATCATGCAATAAT
SEQ ID NO: 16 (16S Probe)
GCGTAACGCGTATGCAACTTGCCTTAC
SEQ ID NO: 17 (16S GBlock)
GAGTTTGATTCTGGCTCAGGATGAACGCTAGCGATAGGCTTAACACATGC
AAGTCGAGGGGCAGCATGAACTTAGCTTGCTAAGTTTGATGGCGACCGGC
GCACGGGTGCGTAACGCGTATGCAACTTGCCTTACAGAGGGGGATAACCC
GTTGAAAGACGGACTAATACCGCATACACTTGTATTATTGCATGATATTA
CAAGGAAATATTTATAGCTGTAAGATAGGCATGCGTCCCATTAGCTGGTT
GGTGAGGTAACGGCTCACCAAGGCAACGATGGGTAGGGGAACTGAGAGGT
SEQ ID NO: 18 (Kgp For1.2)
CTGCACTGTAATACAAGTCG
SEQ ID NO: 19 (Kgp Rev1.2)
CTCAAGCCTTGGCTCACTTG
SEQ ID NO: 20 (Kgp For1.1)
CAACCAAAGCCAAGAAGA
SEQ ID NO: 21 (Kgp Rev1.1)
CGAAGCTGAAGTAGGAAC
SEQ ID NO: 22 (Kgp Probe)
CACTAGCTGCCAATCCATCATT
SEQ ID NO: 23 (Kgp Gblock)
ATACATTTCAGGGAAATAGTCGCCATCGACTGCACTGTAATACAAGTCGG
TAACTTTTTTTGTTTTCTTTCCTTTTTCTCCGCTAATAACGTCAGTGTCA
CCAACCAAAGCCAAGAAGACCGGAGCAGCACTAGCTGCCAATCCATCATT
GTATTTCTTGTGAATAAATGCCTTGATAGAGGCGTTTGTCGTTCCTACTT
CAGCTTCGTCTGTGTAATGCACATCCAGATAGAAGCCCTTTTGAGCCTTC
CAAGTGAGCCAAGGCTTGAGAGCTTCTTTGAATTTTGCACCTGCAACAAC

1. A method for detecting microbial nucleic acid in a body fluid of a subject, the method comprising:
performing an amplification reaction under conditions sufficient to amplify a microbial polynucleotide, and
detecting the amplified microbial polynucleotide, thereby determining that the microbial nucleic acid is present in the body fluid of the subject.

2. The method of claim 1, wherein the body fluid sample is a cerebrospinal fluid sample obtained from the subject, or wherein the body fluid sample comprises DNA purified from a cerebrospinal fluid sample obtained from the subject.

3. (canceled)

4. The method of claim 1, wherein the length of the amplified microbial polynucleotide is less than 400 bases.

5. (canceled)

6. The method of claim 1, wherein the microbial nucleic acid is an oral pathogen nucleic acid.

7. (canceled)

8. The method of claim 1, wherein the microbial nucleic acid comprises P. gingivalis DNA.

9. The method of claim 1, wherein the amplified microbial polynucleotide is a conserved microbial gene segment.

10. The method of claim 9, wherein the conserved microbial gene segment is a P. gingivalis hmuY gene segment or a 16S rDNA segment.

11. (canceled)

12. The method of claim 1, wherein performing the amplification reaction comprises:
(i) combining a body fluid sample with a forward primer, a reverse primer, and a polymerase to form a polymerase chain reaction (PCR) mixture;
(ii) conducting a PCR with the PCR mixture,
wherein the PCR is conducted under conditions sufficient to amplify the microbial polynucleotide.

13. The method of claim 12, wherein the forward primer and the reverse primer are selected such that the length of the amplified microbial polynucleotide is less than 400 bases.

14-25. (canceled)

26. The method of claim 1, further comprising diagnosing a microbial infection in the subject when it is determined that the microbial nucleic acid is present in the body fluid of the subject.

27. The method of claim 26, wherein the microbial infection is a brain infection.

28. The method of claim 26, wherein the microbial infection is a chronic infection.

29. The method of claim 26, further comprising administering an active agent for treating the infection to the subject.

30. The method of claim 29, wherein the active agent is a bacteriocidal agent or a bacteriostatic agent.

31. The method of claim 29, wherein the active agent is a gingipain inhibitor.

32. The method of claim 1, wherein intact microbial cells are not detectable in the body fluid.

33. A kit for detection of microbial nucleic acid in a body fluid sample, the kit comprising one or more components selected from: (i) a set of oligonucleotide primers for amplification of a microbial polynucleotide; and (ii) a detection reagent.

34. The kit of claim 33, wherein the oligonucleotide primers are selected for amplification of a microbial polynucleotide less than 400 bases in length.

35. The kit of claim 34, wherein the microbial polynucleotide is a P. gingivalis gene segment.

36-37. (canceled)

38. The kit of claim 33, for use in the detection of microbial nucleic acid in a cerebrospinal fluid sample.


SEQUENCE LISTING (24 sequences)

SEQ ID NO: 1 - DNA, 651 bp, Porphyromonas gingivalis
atgaaaaaaatcattttctccgcactctgtgcattgccattgattgtgtctctaacttcttgtgggaagaagaaagacgagccgaaccaaccctccacacccgaagcagtaaccaaaaccgtaactatcgatgcttcgaaatacgaaacgtggcagtatttctctttttccaaaggtgaagtcgtaaatgttaccgactataagaacgatttgaactgggacatggctcttcaccgctatgacgttcgtctcaattgtggcgaaagtggtaagggaaaaggtggtgccgtattctccggcaagacagaaatggatcaggctactaccgttccgacagacggatatactgtagatgttctcggccgtattacagtcaagtacgaaatgggacctgatggtcatcagatggaatatgaagaacagggcttcagcgaagtgattaccggcaagaagaacgcacagggatttgcttcaggtggttggctggaattctctcacggtcctgccggtcccacttacaagctgagcaaaagagtcttcttcgttcgtggtgctgatggtaatattgccaaagtgcagttcactgactatcaggatgcagaactcaaaaaaggagtcatcactttcacttatacataccccgttaaataa

SEQ ID NO: 2 - DNA, 20 bp, Artificial Sequence
  Description of Artificial Sequence Synthetic primer
ggtgaagtcgtaaatgttac

SEQ ID NO: 3 - DNA, 20 bp, Artificial Sequence
  Description of Artificial Sequence Synthetic primer
ttgactgtaatacggccgag

SEQ ID NO: 4 - DNA, 20 bp, Artificial Sequence
  Description of Artificial Sequence Synthetic primer
gaacgatttgaactgggaca

SEQ ID NO: 5 - DNA, 20 bp, Artificial Sequence
  Description of Artificial Sequence Synthetic primer
aacggtagtagcctgatcca

SEQ ID NO: 6 - DNA, 15 bp, Artificial Sequence
  Description of Artificial Sequence Synthetic probe
gccggagaatacggc

SEQ ID NO: 7 - DNA, 250 bp, Artificial Sequence
  Description of Artificial Sequence Synthetic polynucleotide
aaacgtggcagtatttctctttttccaaaggtgaagtcgtaaatgttaccgactataagaacgatttgaactgggacatggctcttcaccgctatgacgttcgtctcaattgtggcgaaagtggtaagggaaaaggtggtgccgtattctccggcaagacagaaatggatcaggctactaccgttccgacagacggatatactgtagatgttctcggccgtattacagtcaagtacgaaatgggacctga

SEQ ID NO: 8 - DNA, 19 bp, Artificial Sequence
  Description of Artificial Sequence Synthetic primer
acgagtattgcattgaatg

SEQ ID NO: 9 - DNA, 21 bp, Artificial Sequence
  Description of Artificial Sequence Synthetic primer
accctttaaacccaataaatc

SEQ ID NO: 10 - DNA, 19 bp, Artificial Sequence
  Description of Artificial Sequence Synthetic primer
acgagtattgcattgaatg

SEQ ID NO: 11 - DNA, 21 bp, Artificial Sequence
  Description of Artificial Sequence Synthetic primer
accctttaaacccaataaatc

SEQ ID NO: 12 - DNA, 20 bp, Artificial Sequence
  Description of Artificial Sequence Synthetic primer
aggatgaacgctagcgatag

SEQ ID NO: 13 - DNA, 21 bp, Artificial Sequence
  Description of Artificial Sequence Synthetic primer
gtgagccgttacctcaccaac

SEQ ID NO: 14 - DNA, 20 bp, Artificial Sequence
  Description of Artificial Sequence Synthetic primer
cgaggggcagcatgawctta

SEQ ID NO: 15 - DNA, 20 bp, Artificial Sequence
  Description of Artificial Sequence Synthetic primer
ttgtaatatcatgcaataat

SEQ ID NO: 16 - DNA, 27 bp, Artificial Sequence
  Description of Artificial Sequence Synthetic probe
gcgtaacgcgtatgcaacttgccttac

SEQ ID NO: 17 - DNA, 300 bp, Artificial Sequence
  Description of Artificial Sequence Synthetic polynucleotide
gagtttgattctggctcaggatgaacgctagcgataggcttaacacatgcaagtcgaggggcagcatgaacttagcttgctaagtttgatggcgaccggcgcacgggtgcgtaacgcgtatgcaacttgccttacagagggggataacccgttgaaagacggactaataccgcatacacttgtattattgcatgatattacaaggaaatatttatagctgtaagataggcatgcgtcccattagctggttggtgaggtaacggctcaccaaggcaacgatgggtaggggaactgagaggt

SEQ ID NO: 18 - DNA, 20 bp, Artificial Sequence
  Description of Artificial Sequence Synthetic primer
ctgcactgtaatacaagtcg

SEQ ID NO: 19 - DNA, 20 bp, Artificial Sequence
  Description of Artificial Sequence Synthetic primer
ctcaagccttggctcacttg

SEQ ID NO: 20 - DNA, 18 bp, Artificial Sequence
  Description of Artificial Sequence Synthetic primer
caaccaaagccaagaaga

SEQ ID NO: 21 - DNA, 18 bp, Artificial Sequence
  Description of Artificial Sequence Synthetic primer
cgaagctgaagtaggaac

SEQ ID NO: 22 - DNA, 22 bp, Artificial Sequence
  Description of Artificial Sequence Synthetic probe
cactagctgccaatccatcatt

SEQ ID NO: 23 - DNA, 300 bp, Artificial Sequence
  Description of Artificial Sequence Synthetic polynucleotide
atacatttcagggaaatagtcgccatcgactgcactgtaatacaagtcggtaactttttttgttttctttcctttttctccgctaataacgtcagtgtcaccaaccaaagccaagaagaccggagcagcactagctgccaatccatcattgtatttcttgtgaataaatgccttgatagaggcgtttgtcgttcctacttcagcttcgtctgtgtaatgcacatccagatagaagcccttttgagccttccaagtgagccaaggcttgagagcttctttgaattttgcacctgcaacaac

SEQ ID NO: 24 - DNA, 840 bp, Porphyromonas gingivalis
taaggtcagataattatgaaaaaaatcattttctccgcactctgtgcattgccattgattgtgtctctaacttcttgtgggaagaagaaagacgagccgaaccaaccctccacacccgaagcagtaaccaaaaccgtaactatcgatgcttcgaaatacgaaacgtggcagtatttctctttttccaaaggtgaagtcgtaaatgttaccgactataagaacgatttgaactgggacatggctcttcaccgctatgacgttcgtctcaattgtggcgaaagtggtaagggaaaaggtggtgccgtattctccggcaagacagaaatggatcaggctactaccgttccgacagacggatatactgtagatgttctcggccgtattacagtcaagtacgaaatgggacctgatggtcatcagatggaatatgaagaacagggcttcagcgaagtgattaccggcaagaagaacgcacagggatttgcttcaggtggttggctggaattctctcacggtcctgccggtcccacttacaagctgagcaaaagagtcttcttcgttcgtggtgctgatggtaatattgccaaagtgcagttcactgactatcaggatgcagaactcaaaaaaggagtcatcactttcacttatacataccccgttaaataagttaagagggaaatatgaaaagtgtagtaacaaagcaggccctcatcggcctgcttttctttagtataagtatatactcccatgcggccaaccctccggcccaacctaccgacaccatcgtatccggcaatatcgcacttgaggatatagtggtgaccggtagccgtacagccc